SEQUENCE LISTING (9 sequences)

SEQ ID NO: 1 - protein, 393 aa, HCV
MSTNPKPQRKTKRNTNRRPQDVKFPGGGQIVGGVYLLPRRGPRLGVRATRKTSERSRSMETTMRSPVFTDNSSPPAVPQTFQVAHLHAPTGSGKSTKVPAAYAAQGYKVLVLNPSVAATLGFGTYMSKAHGVDPNIRTGVRTITTGAPITYSTYGKFLADGGCSGGAYDIIICDECHSTDSTSILGIGTVLDQAETAGARLVVLATATPPGSVTVPHPNIEEVGLSNTGEIPFYGKAIPIEAIKGGRHLIFCHSKKKCDELAAKLSGLGLNAVAYYRGLDVSVIPTSGDVVVVATDALMTGYTGDFDSVIDCNTCVTQTVDFSLDPTFTIETTTVPQDAVSRSQRRGRRSLPAILSPGALVVGVVCAAILRRHVGPGEGAVQWMNRLIAFASR

SEQ ID NO: 2 - protein, 277 aa, HCV
MSTNPKPQRKTKRNTNRRPQDVKFPGGGQIVGGVYRSQTFQVAHLHAPTGSGKSTKVPAAYAAQGYKVLVLNPSVAATLGFGAYMSKAHGIDPNIRTGVRTITTGGPVTYSTYGKFLADGGCSGGAYDIIICDECHSTDSTSILGIGTVLDQAETAGARLVVLATATPPGSVTVPHPNIEEVALSNTGEIPFYGKAIPIEVIKGGRHLIFCHSKKKCDELAAKLSGLGLNAVAYYRGLDVSRSSPGALVVGVVCAAILRRHVGPGEGAVQWMNRLIA

SEQ ID NO: 3 - protein, 175 aa, HCV
MSTNPKPQRKTKRNTNRRPQDVKFPGGGQSVGGVYLLPRRGPRLGVRATRKTSERSQPRGRRQPIPKARRPEGRTWAQPGYPWPLYGNEGMGWAGWLLSPRGSRPSWGPSDPRRRSRNLGKVIDTLTCGFADLMGYIPVVGAPLGGAARALAHGVRVLEDGVNYATGNLPGCSFS

SEQ ID NO: 4 - DNA, 30 bp, artificial
  W135-F
cgcggatccatgtctaccaacccgaaaccg

SEQ ID NO: 5 - DNA, 29 bp, artificial
  W135-R
ccggaattcacgagaagcgaaagcgatca

SEQ ID NO: 6 - DNA, 30 bp, artificial; identical to SEQ ID NO: 4
  W102-F

SEQ ID NO: 7 - DNA, 31 bp, artificial
  W102-R
ccggaattcagcgatcagacggttcatccac

SEQ ID NO: 8 - DNA, 30 bp, artificial; identical to SEQ ID NO: 4
  C175-F

SEQ ID NO: 9 - DNA, 29 bp, artificial
  C175-R
ccggaattcagagaaagagcaacccggca

(19) United States
(12) Patent Application Publication
(10) Pub. No.: US 2022/0252614 A1
(43) Pub. Date: Aug. 11, 2022

(54) Hepatitis C Virus Detection Kit

(71) Applicant: Fapon Biotech Inc., Shenzhen, Guangdong (CN)

(72) Inventors: Shaoli PAN, Dongguan, Guangdong (CN); Ruijing LI, Dongguan, Guangdong (CN); Xiuling YU, Dongguan, Guangdong (CN); Zhenzhu CHENG, Dongguan, Guangdong (CN); Yunbo WU, Dongguan, Guangdong (CN); Yichen CHEN, Dongguan, Guangdong (CN); Langshan CHI, Dongguan, Guangdong (CN); Hang GONG, Dongguan, Guangdong (CN); Suiyan OUYANG, Dongguan, Guangdong (CN)

(21) Appl. No.: 17/606,059

(22) PCT Filed: Apr. 23, 2020
(86) PCT No.: PCT/CN2020/086437

§ 371 Date: Oct. 25, 2021

(30) Foreign Application Priority Data
Apr. 30, 2019 (CN) 201910367283.6

(51) Int. Cl. G01N 33/68; G01N 33/576

(52) U.S. Cl. CPC G01N 33/6854; G01N 2469/10; G01N 33/5767

(57) ABSTRACT

Provided is a hepatitis C virus detection kit, a method for detecting hepatitis C virus, and a method for preparing a reagent or kit for detecting the hepatitis C virus. The kit contains a primary antibody and a second antibody for detecting a hepatitis C virus core antigen, wherein the primary antibody is directed against an epitope in a 95th-117th amino acid sequence of the hepatitis C virus core antigen or specifically binds to the 95th-117th amino acid sequence of the hepatitis C virus core antigen; and the second antibody is directed against an epitope in a 55th-72nd amino acid sequence of the hepatitis C virus core antigen or specifically binds to the 55th-72nd amino acid sequence of the hepatitis C virus core antigen. The kit has high sensitivity, good stability, and simple operation, and can be used for rapid detection of early acute hepatitis C.

### CROSS-REFERENCE TO RELATED APPLICATION

The present application is a National Stage of International Patent Application No: PCT/CN2020/086437 filed on Apr. 23, 2020, which claims the benefit of the priority of Chinese Patent Application No. 201910367283.6 filed to the China National Intellectual Property Administration on Apr. 30, 2019, and titled “hepatitis C virus detection kit”; the entire content of which is incorporated in the present application by reference.

### SEQUENCE LISTING

The present application contains a Sequence Listing which has been submitted electronically in ASCII format and is hereby incorporated by reference in its entirety. Said ASCII copy is named_Sequence_Listing.txt and is 8.3 kilobytes in size, and contains 9 sequences from SEQ ID NO:1 to SEQ ID NO:9 which are identical to the sequence listing filed in the corresponding international application NO. PCT/CN2020/086437 filed on Apr. 23, 2020.

### TECHNICAL FIELD

The present disclosure relates to the field of virus detection. Specifically, the present disclosure relates to a method and kit for detecting hepatitis C virus.

### BACKGROUND

Hepatitis C is one of the infectious diseases seriously threatening human health; and at present, there is no effective vaccine to prevent its propagation. The emergence of direct-acting antiviral agents (DAAs) greatly improves the therapeutic effect of hepatitis C, but DAAs have not come into the market in China currently. At the present stage, the major antiviral therapy to the (hepatitis C virus) HCV infected is still a therapeutic regimen based on interferon in China. Compared with hepatitis B in clinical manifestation, hepatitis C has mild symptoms, less severe patients, and develops slowly. Therefore, it is not easy to catch the attention of clinician and patients. HCV is mainly transmitted by blood transfusion and blood products, and may cause acute or chronic infection. Acute HCV infection usually has no symptom, and causes a life-threatening disease only in very rare circumstances. About 15%-45% of the infected can automatically eliminate the virus within 6 months after infection without any treatment. The rest 60%-80% of the infected will lead to chronic hepatitis C virus infection. In these chronic HCV infected people, the risk probability of occurring liver cirrhosis within 20 years is 15%-30%. Hepatitis C has a big difficulty in treatment, long course of treatment, poor therapeutic effect and high cost. Therefore, it is very important to choose an ideal detection method, thus detecting HCV as soon as possible.

HCV is a kind of spherical coated positive-sense single-stranded RNA virus having a total length of about 9500 bases, and belongs to the flavivirus family. Both sides of the HCV genome are 5′ and 3′ noncoding regions, and the middle part is Open Reading Frame (ORF), divided into a structural region and a non-structural region. The structural region includes a core protein region (C) and two envelope protein regions (E1, E2), which respectively encode core proteins and envelope proteins. The non-structural protein region includes regions NS2, NS3, NS4 and NS5, encoding functional proteins, such as, protease (regions NS2, NS3 and NS4A), helicase (NS3) and RNA-dependent RNA polymerase (NS5B region). HCV core protein contains about 190aa and plays a very important role in virus replication. The above structural and non-structural proteins are usually expressed by genetic engineering as an envelope antigen to construct an ELISA method for anti-HCV detection. HCV genome has significant heterogeneity, and the degree of variation in the same genome significantly varies from the difference of the regions. The 5′ noncoding region is the most conservative, and has become the research focus on the HCV molecular diagnosis.

At present, method for detection of HCV mainly includes three types: HCV antibody testing, HCV core antigen testing, and HCV nucleic acid testing of hepatitis C virus RNA. HCV antibody testing is the most common method to judge and screen whether a patient is infected with HCV for hospitals and blood stations at present, but has a critical defect of “window phase”, that is, there is a time period of 40 d-70 d between HCV infection and the production of HCV antibodies; during such period, if the blood donor has been infected and infectious, the virus cannot be detected with the current antibody detection reagent. The period is called Preseroconversion Window Phase (PWP). The existence of PWP is the major reason of transfusion infection. Currently, posttransfusion infection of hepatitis C accounts for 70% of the hepatitis cases, and 80%-90% of the HCV infected people belong to posttransfusion infection. HCV core antigens will produce in the body of the infected within 1-2 d after the production of HCV nucleic acid, and has certain correlation with the degree of HCV nucleic acid and thus, may be used as a marker to detect HCV. HCV nucleic acid testing (NAT) is the most reliable in the three test methods; NAT can detect HCV nucleic acid in the early stage of the infection, and can reflect the content of the virus and thus, is mainly used for the selection of antiviral therapy and efficacy monitoring. But NAT needs to be operated in strict accordance with PCR operating procedures, and the testers need to receive professional training and acquire corresponding qualifications; moreover, the sample demands for high quality control, namely, samples must be sent for testing at low temperature within 2 h after blood sampling, and RNA is extracted under sterile conditions. Therefore, the method is easy to cause an error due to the operation, equipment, environment and other factors, thereby producing false positive or false negative, which is against the promotion in general hospitals and has smaller market share.

HCV antigen-antibody combination detection may detect the HCV antigen and antibody in a sample simultaneously. However, in the HCV antigen-antibody combination detection, antigens and antibodies must be subjected to a large number of screening and experiments, thus avoiding the overlapped epitope of the antigens and antibodies, and the cross reaction between anti-HCV antigen monoclonal antibodies and HCV recombinant antigens. In the HCV antigen-antibody combination detection, the selected antigen region must further possess high immunogenicity, thus facilitating the preparation of antibodies and antigen capture in a sample. In prior arts, a known epitope binding domain of a monoclonal antibody in a core antigen should be subjected to mutation or deletion, such that the monoclonal antibody for detecting the HCV core antigen will not bind to these mutative and deleted core antigens, but still bind to the intact core antigens from the sample. For example, CN105228649A discloses a mutant core protein antigen comprising a deletion of amino acids 34 and 48 and amino acids 115-121 for combination assay; and further discloses a deletion of 5 amino acids (32, 33 and 34 for the C11-9 binding region and amino acid residues 47 and 48 from the C11-14 binding region of core) to obviate the problem of the reaction between core antigens used for the capture of core antibodies and detection antibodies used for the detection of core antigens. However, these constructs yielded poorer anti-core antibody detection as these deleted residues are highly immunogenic in patients (see CN105228649A). Therefore, it is advantageously that the selected antigen domains are not detected by detection antibodies, but preserve or enhance the detection to the anti-core antibody samples. The prior art CN1489692A discloses an HCV antigen/antibody combination assay, and teaches that HCV core antigens, such as amino acids 10-53 and 120-130 are in combination with NS3 antibodies for detection. Such combination assay usually performs a large number of cross-over experiments and screening on epitope of antibodies for HCV antigens of a patient to be captured and HCV antibodies of a captured patient by using an antigen interval, which demands for higher labor, instrument and reagent costs.

The antigen-antibody combination assays on the current market are comparatively limited to ELISA, plate fluorescence and time resolution; and these methodologies have the disadvantages of long reaction time, high consumption of manpower and material resources, and increased cost.

### SUMMARY

Through a lot of theoretical studies and experimental exploration, the inventor fully considers the whole process of HCV infection to analyze and study antibodies used for capturing HCV antigens of a patient and antigen regions used for capturing HCV antibodies, and obtain an HCV core antigen region combination capable of being used for detecting HCV through a large number of experiments and screening. The disclosure has proved that the selected epitope region has excellent immunogenicity, and antibodies prepared thereby unexpectedly can be combined with each other for high-activity detection of HCV core antigens. Surprisingly, the inventor further finds that monoclonal antibodies prepared by the antigen may combine HCV antigens to mutually supplement the shortage in the single detection of HCV antigens or antibodies, thereby reducing the risk of missing detection and shortening the window phase.

Therefore, in some embodiments, the present disclosure provides a hepatitis C virus detection kit and a preparation method. The kit of the present disclosure has improved sensitivity and stability, shortened reaction time, easy operation, suitable for popularization and disclosure. The kit of the present disclosure especially shortens the window phase and reaction time and thus, can be used for the rapid diagnosis of acute hepatitis C in early stage. In some embodiments, the hepatitis C virus detection kit provided by the present disclosure includes a primary antibody and a second antibody for detecting a hepatitis C virus core antigen, where the primary antibody is directed against an epitope in 95th-117th amino acid sequence of the hepatitis C virus core antigen; and the second antibody is directed against an epitope in 55th-72nd amino acid sequence of the hepatitis C virus core antigen. In some embodiments, the primary antibody specifically binds to the 95th-117th amino acid sequence of the hepatitis C virus core antigen. In some embodiments, the second antibody specifically binds to the 55th-72nd amino acid sequence of the hepatitis C virus core antigen. In some embodiments, the primary antibody and/or the second antibody may be a monoclonal antibody. In some embodiments, antibodies of the present disclosure are prepared by a method known in the art, for example, the primary antibody and/or the second antibody. In some embodiments, an animal may be immunized by an antigen containing the 55th-72nd amino acid sequence and/or an antigen containing the 95th-117th amino acid sequence to prepare the antibodies of the present disclosure, for example, the primary antibody and/or the second antibody. In some embodiments, the 55th-72nd amino acid sequence and/or the 95th-117th amino acid sequence may serve as an antigen to immunize an animal to prepare the antibodies of the present disclosure, for example, the primary antibody and/or the second antibody. In some embodiments, when used herein, the “specific binding” may refer to that an antibody selectively or preferably binds to the amino acid sequence. A standard assay, e.g., plasmon resonance technology (for example, BIACORE®) may be used to determine binding affinity. In some embodiments, the primary antibody binds to the same epitope as an antibody specifically binding to 95th-117th amino acid sequence of the hepatitis C virus core antigen. In some embodiments, the second antibody binds to the same epitope as an antibody specifically binding to 55th-72nd amino acid sequence of the hepatitis C virus core antigen. The “antibody binding to the same epitope” as a reference antibody refers to that, for example, above 50% binding of the reference antibody to the antigen thereof is blocked in competitive immunometric assay; or above 50% binding of the antibody to the antigen thereof is blocked in competitive immunometric assay via the reference antibody.

In some embodiments, any suitable in vitro assay, cell-based assay, in vitro assay, animal models and the like may be used for detecting the effects of the antibodies in the present disclosure, such as binding activity and/or cross-reactivity. In some embodiments, the assay may include, for example, ELISA, FACS binding assay, Biacore, competitive binding assay, and the like. In some embodiments, for example, in ELISA or FACS, an EC50 value of the binding of the antibodies (or antigen-binding fragments thereof) in the present disclosure to antigens may be, for example, 1 μM-1 pM, for example, 1 nM-1 pM, for example, 100 pM-1 pM.

In some embodiments, the primary antibody and the second antibody in the kit are free of cross reaction. In some embodiments, the primary antibody (directed against the epitope in 95th-117th amino acids) and the second antibody (directed against the epitope in 55th-72nd amino acids) may serve as a capture antibody (or called an envelope antibody) and a labeled antibody, for example, the primary antibody is a capture antibody and the second antibody is a labeled antibody; or the primary antibody is a labeled antibody and the second antibody is a capture antibody. Preferably, in some embodiments, the primary antibody is a capture antibody (or called an envelope antibody), and the second antibody is a labeled antibody. In some embodiments, an alternative antibody may further serve as an envelope antibody or a labeled antibody. For example, in some embodiments, the antibody directed against the epitope in 17th-35th amino acids may serve as a capture antibody (or called an envelope antibody).

In some embodiments, the capture antibody is bound to a solid phase. In some embodiments, the capture antibody may be used to coat a solid phase support. In some embodiments, the solid phase support is not limited particularly, and may be, for example, magnetic particles, e.g., a magnetic bead, latex particle and a microtitration plate. In some embodiments, the labeled antibody is labeled by a detectable label, for example, labeled by a fluorescent label, e.g., acridinium ester, for example, labeled by a fluorescent label, e.g., acridinium ester via an adapter, e.g., biotin-avidin.

In some embodiments, the term “antibody” in the present disclosure may be used in the broadest sense; it may include full-length monoclonal antibodies, bispecific or multispecific antibodies, chimeric antibodies, and antigen-binding fragments of the antibodies as long as these antibodies show required bioactivities, e.g., specific binding to HCV antigens. The “antibody fragment” includes a portion of the full-length antibody, preferably, an antigen binding region or a variable region thereof. Examples of the antibody fragment include Fab, Fab′, F(ab′)₂, Fd, Fv, dAb, a complementary determining region (CDR) fragments, single-chain antibodies (e.g., scFv), bivalent antibodies or binding domain antibodies.

In some embodiments, the kit further includes a primary antigen and a second antigen for detecting a hepatitis C virus antibody in a sample from a subject. In some embodiments, the primary antigen and the second antigen may be, for example, hepatitis C virus core antigens, E1, E2, NS2, NS3, NS4 [Mimms et al., Lancet 336:1590 (1990); Bresters et al., Vox Sang 62:213 (1992)] and NS5. In some embodiments, the primary antigen and the second antigen originate from different positions of a same antigen. In some embodiments, the primary antigen and the second antigen may be selected from antigens as shown in SEQ ID NO:1 and SEQ ID NO: 2 or immunogenic fragments thereof. For example, in some embodiments, the primary antigen and the second antigen may be 1st-56th amino acids of an HCV core antigen, 1201st-1490th amino acids of an NS3, a 1883rd-1925th amino acid sequence of an NS4; 1st-35th amino acids of the HCV core antigen, 1223rd-1426th amino acids of the NS3, and a 1890th-1923rd amino acid sequence of the NS4.

In some embodiments, the primary antigen and the second antigen may serve as a capture antigen and a labeled antigen, for example, the primary antigen is a capture antigen and the second antigen is a labeled antigen; or the primary antigen is a labeled antigen and the second antigen is a capture antigen. In some embodiments, the primary antigen is a capture antigen, and the second antigen is a labeled antigen.

In some embodiments, the capture antigen is bound to a solid phase. In some embodiments, the capture antigen may be used to coat a solid phase support. In some embodiments, the solid phase support is not limited particularly, and may be, for example, magnetic particles, e.g., a magnetic bead, latex particle and a microtitration plate. In some embodiments, the labeled antigen is labeled by a detectable label, for example, labeled by a fluorescent label, e.g., acridinium ester, for example, the antibody is labeled by a fluorescent label, e.g., acridinium ester via an adapter, e.g., biotin-avidin. In some embodiments, the detectable label for labeling antigens or antibodies is not limited particularly. In some embodiments, the labeling may include, but not limited, fluorescence labeling, chromophore labeling, electron-dense labeling, chemiluminescent labeling, and radiolabeling as well as indirect labeling, e.g., enzyme or ligand, for example, indirect detection is performed by enzymatic reaction or molecular interaction. In some embodiments, exemplary labeling includes, but not limited to, radioisotope, fluorophore, rhodamine and derivatives thereof, luciferase, fluorescein, horse radish peroxidase (HRP), alkaline phosphatase, β-galactosidase, glucoamylase, lysozyme, saccharides oxidases, such as, glucose oxidase, galactose oxidase, and glucose-6-phosphate dehydrogenase, biotin/avidin, spin labeling, phage labeling and the like.

In some embodiments, the kit of the present disclosure includes a regent suitable for performing immunoassay. In some embodiments, the kit of the present disclosure may be used for immunoassay, for example, ELISA, indirect immunofluorescence assay (IFA), radioimmunoassay (RIA), and other tests or methods except enzyme linked immunosorbent assay.

In some embodiments, for example, in the ELISA assay, HCV antibodies may be coated on a solid phase, e.g., a magnetic bead to capture HCV antigens in a sample, and then labeled antibodies bind to antigens bound on a reaction plate again, and the result is read after color developing. In some embodiments, an HCV antibody of the present disclosure may be coated on a solid phase, e.g., a magnetic bead or serve as a labeled second antibody. In some embodiments, antibodies or antigen-binding fragments thereof are fixed onto a surface, for example, onto a solid phase support, for example, plastics, membranes, e.g., nitrocellulose membrane, glass, magnetic beads or metal supports. In some embodiments, the sample from the subject contacts with the solid phase support, and then contact strips are color developed by an antibody indicator of the detectable label. In some embodiments, blocking agents, such as bovine serum albumin (BSA), milk powder solution, gelatin, PVP, Superblock may be used to block non-specific sites, thereby reducing the background caused by non-specific binding. In some embodiments, a diluent may be used, e.g., BSA and phosphate buffer (PBS)/Tween may be used to dilute antiserum, which facilitates the reduction of non-specific background.

In this article, the sample from the subject may include a healthy or pathological biological tissue, cell or body fluid, for example, a blood sample, for example, plasma, serum, blood products, for example, seminal fluid or vaginal secretion.

In some embodiments, the kit further includes a virus lysis solution. In some embodiments, the virus lysis solution may include, for example, denaturants, surfactants, protective proteins, ammonium sulfate and absolute ethyl alcohol. In some embodiments, the virus lysis solution may be a buffer, for example, a phosphate buffer (PBS). In some embodiments, the virus lysis solution requires no dissociation of antigen/antibody, and a mild lysis solution is adjusted, free of influencing the sensitivity of the antibody, which is beneficial to the antigen-antibody binding, and can release the core antigen in the virus, thus achieving the efficient reaction between antibodies and antigens, thereby improving the detection ratio of the virus.

In some embodiments, the present disclosure provides a use of the primary antibody and the second antibody for detecting a hepatitis C virus core antigen in the preparation of a kit for detecting hepatitis C virus. In some embodiments, the present disclosure provides a method of detecting hepatitis C virus, and the method includes contacting the sample from the subject with the primary antibody and the second antibody. In some embodiments, the primary antibody is directed against an epitope in a 95th-117th amino acid sequence of the hepatitis C virus core antigen; and the second antibody is directed against an epitope in a 55th-72nd amino acid sequence of the hepatitis C virus core antigen. In some embodiments, the present disclosure provides a use of a combination of an immunogenic polypeptide containing the 95th-117th amino acids of the hepatitis C virus core antigen and an immunogenic polypeptide containing the 55th-72nd amino acids of the hepatitis C virus core antigen in the preparation of an antibody for detecting a hepatitis C virus core antigen. In some embodiments, the present disclosure provides a method for preparing the antibody for detecting the hepatitis C virus core antigen; the method includes: using the immunogenic polypeptide containing the 95th-117th amino acids of the hepatitis C virus core antigen and the immunogenic polypeptide containing the 55th-72nd amino acids of the hepatitis C virus core antigen to immunize animals respectively, thus preparing antibodies for detecting the hepatitis C virus core antigen, e.g., monoclonal antibodies. In some embodiments, the immunogenic polypeptide includes the 95th-117th amino acids of the hepatitis C virus core antigen and/or an adjuvant, as well as the 55th-72nd amino acids of the hepatitis C virus core antigen and/or an adjuvant. In some embodiments, a core antigen epitope region identified in the present disclosure, such as the 95th-117th amino acids of the hepatitis C virus core antigen and the 55th-72nd amino acids of the hepatitis C virus core antigen (artificially synthesized by, for example, a chemical method) may be linked with a proper carrier protein, which is used to immunize animals to prepare antibodies, e.g., monoclonal antibodies. In some embodiments, the proper carrier protein is known in the art, and may be, for example, KLH and BSA, and the like. In some embodiments, the kit of the present disclosure may include the above primary antibody and second antibody, and may further include the primary antigen and/or second antigen of hepatitis C virus. In some embodiments, the method for detecting hepatitis C virus of the present disclosure may further include: contacting the sample from the subject with the primary antigen and/or the second antigen from hepatitis C virus. In some embodiments, the primary antigen and/or the second antigen may be, for example, a hepatitis C virus core antigen, E1, E2, NS2, NS3 NS4 and NS5, for example, the primary antigen and/or the second antigen originate from different positions of a same hepatitis C virus antigen, for example, a 7th-48th amino acid sequence from the hepatitis C virus core antigen, for example, a 7th-21st amino acid sequence and/or 29th-48th amino acid sequence from the hepatitis C virus core antigen; for example, the primary antigen and/or the second antigen may include any one of the following amino acid fragments or a combination thereof: 1st-56th amino acids of an HCV core antigen, 1201st-1490th amino acids of NS3, a 1883rd-1925th amino acid sequence of NS4; 1st-35th amino acids of the HCV core antigen, 1223rd-1426th amino acids of NS3, a 1890th-1923rd amino acid sequence of NS4; for example, an amino acid sequence as shown in SEQ ID NO:1 and/or SEQ ID NO:2.

In some embodiments, the present disclosure provides a method for preparing a reagent or kit for detecting hepatitis C virus, where a primary hepatitis C virus core antigen and a second hepatitis C virus core antigen are used to prepare antibodies. In some embodiments, the present disclosure provides a use of the primary hepatitis C virus core antigen and the second hepatitis C virus core antigen in the preparation of a reagent or kit for detecting hepatitis C virus. In some embodiments, the primary hepatitis C virus core antigen may include or consist of 55th-72nd amino acids of the hepatitis C virus core antigen; and the second hepatitis C virus core antigen may include or consist of 95th-117th amino acids of the hepatitis C virus core antigen. In some embodiments, the kit of the present disclosure may include the above primary hepatitis C virus core antigen and the second hepatitis C virus core antigen, and may further include an antibody, e.g., a monoclonal antibody, directed against one or two antigens in different positions (namely, the positions different from the above primary core antigen and the second core antigen) of hepatitis C virus. In some embodiments, the one or two antigens in different positions of hepatitis C virus may be, for example, a hepatitis C virus core antigen, E1, E2, NS2, NS3 NS4 and NS5, for example, different positions from a same hepatitis C virus antigen, for example, a 7th-48th amino acid sequence from the hepatitis C virus core antigen, for example, a 7th-21st amino acid sequence and/or 29th-48th amino acid sequence from the hepatitis C virus core antigen; for example, may include any one of the following amino acid fragments or a combination thereof: 1st-56th amino acids of an HCV core antigen, 1201st-1490th amino acids of NS3, a 1883rd-1925th amino acid sequence of NS4; 1st-35th amino acids of an HCV core antigen, 1223rd-1426th amino acids of NS3, a 1890th-1923rd amino acid sequence of NS4; for example, an amino acid sequence as shown in SEQ ID NO:1 and/or SEQ ID NO:2. In some embodiments, the primary hepatitis C virus core antigen and the second hepatitis C virus core may be used to prepare an antibody, e.g., a monoclonal antibody. In some embodiments, the kit of the present disclosure may include the antibody, e.g., a monoclonal antibody, prepared by the above primary hepatitis C virus core antigen and second hepatitis C virus core antigen, optionally, may further include one or two antigens in other positions of hepatitis C virus. In some embodiments, the one or two antigens in different positions of hepatitis C virus may be, for example, a hepatitis C virus core antigens, E1, E2, NS2, NS3, NS4 and NS5, for example, different positions from a same hepatitis C virus antigen, for example, a 7th-48th amino acid sequence from the hepatitis C virus core antigen, for example, a 7th-21st amino acid sequence and/or 29th-48th amino acid sequence from the hepatitis C virus core antigen; for example, may include any one of the following amino acid fragments or a combination thereof: 1st-56th amino acids of an HCV core antigen, 1201st-1490th amino acids of NS3, a 1883rd-1925th amino acid sequence of NS4; 1st-35th amino acids of an HCV core antigen, 1223rd-1426th amino acids of NS3, a 1890th-1923rd amino acid sequence of NS4; for example, an amino acid sequence as shown in SEQ ID NO:1 and/or SEQ ID NO:2.

In some embodiments, the present disclosure provides a method, use and a related kit for detecting at least one HCV antigen and at least one HCV antibody simultaneously. In some embodiments, the method may include the following steps: contacting the sample with at least one HCV antigen coated on the solid phase or a fragment thereof to form an immune complex, and simultaneously contacting the sample with at least one HCV antibody coated on the solid phase and/or a fragment thereof to form an immune complex; and detecting the existence of the complex, thereby determining the existence of the HCV antigen and/or antibody in the sample. In some embodiments, the method may include the following steps: contacting the sample with at least one HCV antigen coated on the solid phase or a fragment thereof to form an immune complex, and simultaneously contacting the sample with at least one HCV antibody coated on the solid phase and/or a fragment thereof to form an immune complex; adding a second HCV antigen linked with the detectable label, and a second HCV antibody linked with the detectable label to the produced complex; and detecting the production signal, thus determining the existence of the HCV antigen and/or antibody in the sample. In some embodiments, the present disclosure provides a kit for the method, including 1) a container containing at least one HCV antigen coated on the solid phase, 2) container containing at least one HCV antibody coated on the solid phase, or container containing at least one HCV antigen coated on the solid phase and at least one HCV antibody coated on the solid phase. In some embodiments, the kit further includes a second antigen and/or a second HCV antibody linked with the detectable label. In some embodiments, the at least one HCV antibody coated on the solid phase and the at least one HCV antigen coated on the solid phase are free of cross reaction. In some embodiments, the at least one HCV antibody is a monoclonal antibody of an HCV core antigen. In some embodiments, the at least one HCV antigen is an HCV core antigen, for example, a recombinant antigen. In some embodiments, the HCV core antigen is exclusive of an epitope bound to the antibody, for example, exclusive of the epitope in the 95th-117th amino acid sequence of the core antigen and the epitope in the 55th-72nd amino acid sequence of the core antigen.

In some embodiments, the method and/or use of the present disclosure are free of performing antigen/antibody dissociation. In some embodiments, in the method and/or use of the present disclosure, the primary step may be antibody reaction, beneficial to the preferred binding of antigens to antibodies, and the second step is to add a lysis solution to release the core antigen in the virus, thus achieving the efficient reaction of antigens and antibodies, and improving the detection rate of the virus.

In some embodiments, the primary antigen and the second antigen may serve as a capture antigen and a labeled antigen, for example, the primary antigen is a capture antigen and the second antigen is a labeled antigen; or the primary antigen is a labeled antigen and the second antigen is a capture antigen. In some embodiments, the primary antigen is a capture antigen, and the second antigen is a labeled antigen.

In some embodiments, the present disclosure provides a kit for antigen-antibody combination detection via a magnetic bead. In some embodiments, the kit of the present disclosure may include a reagent suitable for chemiluminescence detection by mechanical energy. In some embodiments, the kit of the present disclosure may make use of an automatic chemiluminiscence instrument to achieve the high-throughput, fast and accurate detection of antigens and antibodies in HCV, which shortens the detection time, and can rapidly detect the results.

In some embodiments, the present disclosure provides a kit for antigen-antibody combination detection via a magnetic bead, including antigens and antibodies labeled on the magnetic bead. In some embodiments, the kit of the present disclosure may utilize a magnetic bead as a solid phase; antigens and antibodies are directly labeled on the magnetic bead to detect the antigens and antibodies in HCV by using a double-antigen sandwich method and a double-antibody sandwich method, which improves the detection rate and shortens the window phase.

In some embodiments, the kit of the present disclosure detects the HCV core antigen, significantly shortening the window phase, ahead of 50 d around averagely, thereby reducing the risk of HCV infection within the window phase.

In some embodiments, in combination with the combination detection of HCV core antigens and HCV antibodies, the present disclosure can overcome the shortage in the single detection of HCV antigens or antibodies, which remarkably shortens the window phase, reduces the risk of missing detection and workload, and lowers the cost of manpower, instrument and reagent of the two methodologies when used for single detection.

In some embodiments, the present disclosure provides a kit for antigen-antibody combination detection of hepatitis C virus via a magnetic bead and a preparation method thereof to solve the problem existing in the prior art; and the problem includes low sensitivity, poor stability, long reaction time and/or complex operation and other technical problems. In some embodiments, the present disclosure shortens the window phase and reaction time and thus, can be used for the rapid diagnosis of acute hepatitis C in early stage.

In some embodiments, the present disclosure may use a magnetic bead as a carrier to detect antigens and antibodies. In some embodiments, the present disclosure may make use of an automatic chemiluminiscence instrument to rapidly and accurately detect the antigens and antibodies in HCV. In some embodiments, a double-antibody sandwich theory may be used to prepare the kit. For example, in some embodiments, antibodies in a sample are firstly captured by a hepatitis C virus recombinant antigen AgI (HCV-AgI) labeled on the magnetic bead and a biotinylated hepatitis C virus recombinant antigen AgII (HCV-AgII-BIO), thus forming a double-antigen sandwich state. In some embodiments, after a lysis solution is added in the sample to lyse hepatitis C virus to obtain a core antigen, the core antigen is captured by a hepatitis C virus monoclonal antibody AbI (HCV-AbI) labeled on the magnetic bead and a biotinylated hepatitis C virus monoclonal antibody AbII (HCV-AbII-BIO), thus forming a double-antibody sandwich state, then other components in the sample are washed. In some embodiments, an avidinylated label SA-AE may be further added to form a monoclonal antibody AbI-hepatitis C virus antigen-biotinylated monoclonal antibody AbII-avidinylated label SA-AE and a recombinant antigen AgI-hepatitis C virus antibody-biotinylated recombinant antigen II-avidinylated label SA-AE. In some embodiments, the plate was washed by buffer with triggers, and an automatic chemiluminiscence instrument is used to measure a luminance value, and the luminance value is in positive correlation to the total concentration of antigens and antibodies in the sample, and compared to the critical value, thus judging as positive or negative.

In some embodiments, the detection antigens and antibodies of the present disclosure are two anti-HCV antigen monoclonal antibodies (AbI and AbII) and two HCV recombinant antigens (AgI and AgII) obtained by analyzing the hepatitis C virus sequence. Moreover, the two anti-HCV antigen monoclonal antibodies and the two HCV recombinant antigens are free of cross reaction. In some embodiments, the anti-HCV antigen monoclonal antibody AbI and HCV recombinant antigen AgI may be used as raw materials for coating the magnetic bead; and the anti-HCV antigen monoclonal antibody AbII and HCV recombinant antigen AgII may be used as biotinylated raw materials; a double-antigen sandwich method is used to detect HCV antigens and a double-antibody sandwich method is used to detect HCV antibodies.

The present disclosure discloses sequences of HCV-AgI and HCV-AgII antigens.

Sequence of the HCV-AgI antigen: SEQ ID NO: 1 (W135)
MSTNPKPQRKTKRNTNRRPQDVKFPGGGQIVGGVYLLPRRGPRLGVRATRKTSERSRSMETTM
RSPVFTDNSSPPAVPQTFQVAHLHAPTGSGKSTKVPAAYAAQGYKVLVLNPSVAATLGFGTYMSKA
HGVDPNIRTGVRTITTGAPITYSTYGKFLADGGCSGGAYDIIICDECHSTDSTSILGIGTVLDQAETA
GARLVVLATATPPGSVTVPHPNIEEVGLSNTGEIPFYGKAIPIEAIKGGRHLIFCHSKKKCDELAAKLS
GLGLNAVAYYRGLDVSVIPTSGDVVVVATDALMTGYTGDFDSVIDCNTCVTQTVDFSLDPTFTIETT
TVPQDAVSRSQRRGRRSLPAILSPGALVVGVVCAAILRRHVGPGEGAVQWMNRLIAFASR
Sequence of the HCV-AgII antigen: SEQ ID NO: 2 (W102)
MSTNPKPQRKTKRNTNRRPQDVKFPGGGQIVGGVYRSQTFQVAHLHAPTGSGKSTKVPAAY
AAQGYKVLVLNPSVAATLGFGAYMSKAHGIDPNIRTGVRTITTGGPVTYSTYGKFLADGGCSGGAY
DIIICDECHSTDSTSILGIGTVLDQAETAGARLVVLATATPPGSVTVPHPNIEEVALSNTGEIPFYGKAI
PIEVIKGGRHLIFCHSKKKCDELAAKLSGLGLNAVAYYRGLDVSRSSPGALVVGVVCAAILRRHVGP
GEGAVQWMNRLIA
C175 sequence (SEQ ID NO: 3)
MSTNPKPQRKTKRNTNRRPQDVKFPGGGQSVGGVYLLPRRGPRLGVRATRKTSERSQPRG
RRQPIPKARRPEGRTVVAQPGYPWPLYGNEGMGWAGWLLSPRGSRPSWGPSDPRRRSRNLGK
VIDTLTCGFADLMGYIPVVGAPLGGAARALAHGVRVLEDGVNYATGNLPGCSFS

The present disclosure discloses a method for preparing an HCV-core antigen McAb.

In some embodiments, the present disclosure may further include: directly labeling an acridinium ester on the HCV-AbII antibody.

In some embodiments, the present disclosure may use a magnetic bead as a solid phase carrier.

In some embodiments, the present disclosure provides an in vitro labeling method of a biotinylated HCV antigen and antibody.

In some embodiments, the present disclosure provides an in vitro labeling method of a biotinylated HCV antigen and antibody with a magnetic bead as a carrier.

In some embodiments, the present disclosure provides a lysis solution for HCV antigen-antibody combination detection; and the lysis solution requires no dissociation of antigens/antibodies, and a mild lysis solution is adjusted, free of influencing the sensitivity of the antibody, which is beneficial to the antigen-antibody binding, and can release the core antigen in the virus, thus achieving the efficient reaction between antibodies and antigens, thereby improving the detection ratio of the virus.

In some embodiments, the present disclosure may use avidinylated or biotinylated SA-AE for labeling.

In some embodiments, the antibody of the present disclosure is directed against an epitope 95-117aa of the core antigen or specifically binds to the sequence. In some embodiments, the labeled antibody is preferably directed against an epitope 55-72aa or specifically binds to the sequence. In some embodiments, the envelope antibody is preferably directed against an epitope 95-117aa or specifically binds to the sequence. In some embodiments, the envelope antigen and antibody are free of cross reaction, and the epitope is not overlapped, thus avoiding the difficult situation that epitopes are difficultly staggered for the previous core antigens and antibodies, resulting in the failure of the preparation of a kit for the combined detection of antigens and antibodies. Moreover, there is loss of activity after mutation in the method of avoiding cross reaction with antibodies by making a mutation on the core antigen epitope. Antigens and antibodies of the present disclosure are not overlapped in epitopes, free of influencing the activity. In some embodiments, a labeled antibody and an envelope antibody of the present disclosure are directed against a paired epitope for the HCV antigen-antibody combination detection, which advantageously avoids the problems of cross reaction and loss of detection activity.

In some embodiments, the HCV antigen I and HCV antigen II of the present disclosure may be any proper HCV antigen, for example, a core antigen, E1, E2, NS2, NS3, NS4 and NS5. In some embodiments, the HCV genotype detected in the present disclosure is not limited particularly, for example, may be I/1a, II/1b, Ill/2a, IV/2b, V/3a, and further VI/3b. In some embodiments, the HCV genotype detected in the present disclosure is HCV1b. In some embodiments, based on the distribution of genotypes in different regions, different genotypes of gene segments or a combination of several genotypes of gene segments may be used, while the selected amino acid segment is constant. In some embodiments, the core segment of the present disclosure is analyzed and screened to determine that the core epitope segment is 7-21aa and 29-48aa of the core antigen. In some embodiments, preferably, the core antigen segment of this present disclosure is 7-48a.

The present disclosure advantageously has one or more of the following advantages:

1. In some embodiments, the HCV core antigens and HCV-core antibodies of the present disclosure may be simultaneously used for combination detection, thus avoiding the mutual cross reaction of the core antigens and the core antibodies. The method may further overcome the shortage in the single detection of HCV antigens or antibodies, which remarkably shortens the window phase, reduces the risk of missing detection and workload, and lowers the cost of manpower, instrument and reagents of the two methodologies when used for detection alone.

2. The present disclosure requires no antigen/antibody dissociation, and a mild lysis solution is adjusted, free of influencing the sensitivity of the antibody; the primary step may be antibody reaction, beneficial to the preferred binding of antigens to antibodies, and the second step is to add a lysis solution to release the core antigen in the virus, thus achieving the efficient reaction of antigens and antibodies, and improving the detection rate of the virus.

3. In some embodiments, the combination detection method provided by the present disclosure may simultaneously detect HCV antigens and antibodies, thus solving the problem that the single detection of core antigens and HCV antibodies requires two kits on the market.

### BRIEF DESCRIPTION OF THE DRAWINGS

FIG. 1 shows a schematic diagram of antigen-antibody combination detection in a nonlimiting embodiment of the present disclosure.

FIG. 2:A:PCR amplified product diagram, 1. DNA marker (DL2000); 2. PCR amplified products; B: PCR identification diagram of a PKO-C175C recombinant plasmid; 1. PCR amplified products; 2. PCR amplified products of a PKO-C175 colony.

FIG. 3: Induction expression analysis and purity analysis on a recombination fusion protein. A:1. Protein marker (KD); 2. BL21(DE3) is free of a PKO-C175 expression plasmid; 3. BL21(DE3) contains the PKO-C175 expression plasmid and is induced by non-IPTG; 4. BL21(DE3) contains the PKO-C175 expression plasmid and is induced by IPTG for 4 h. B: 1. Protein marker (KD); 2. BL21(DE3) contains the PKO-C175 expression plasmid and is induced by IPTG for 4 h; 3. Affinity purification of a C175-A sample containing a fusion protein; 4. Sample C175-B after removing the fusion protein via enterokinase digestion.

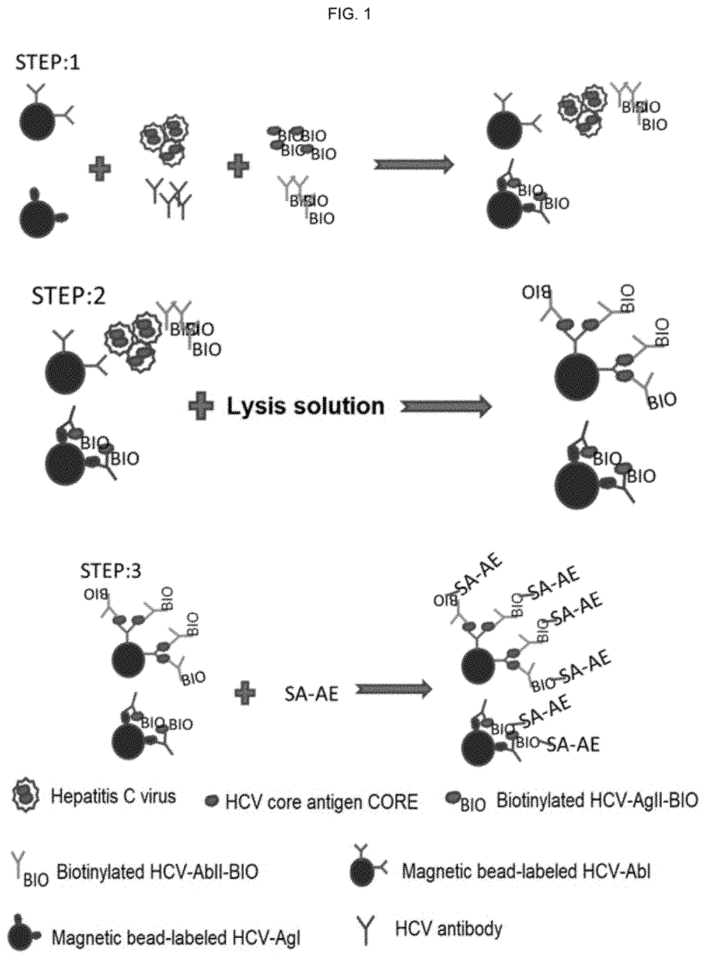

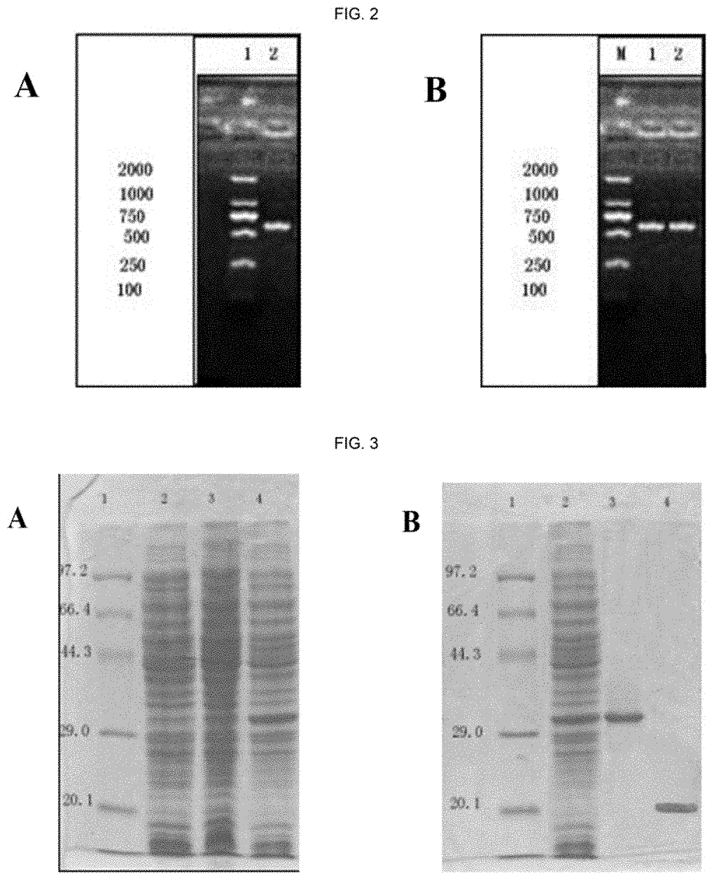

### DETAILED DESCRIPTION OF THE EMBODIMENTS

The kit for the antigen-antibody combination detection of hepatitis C virus via a magnetic bead and a preparation method will be mainly further described specifically with reference to the drawings and specific examples below.

Reagents and Materials:

### Example 1 HCV Antigen Preparation

(1) Preparation of an HCV-AgI envelope antigen: by means of genetic engineering, a lot of molecular biology analysis software were used to analyze and screen out HCV NS3, NS4, and a dominant epitope segment of the core antigen, and the sequence was SEQ ID NO. 1 (named W135); and the codons were optimized, and primers were designed (W135-F (SEQ ID No. 4):CGCGGATCCATGTCTACCAACCCGAAACCG; W135-R (SEQ ID No. 5):CCGGAATTCACGAGAAGCGAAAGCGATCA) to amplify a DNA segment corresponding to W135, and the forward primer respectively carried a BamHI restriction enzyme cutting site, and the reverse primer carried an EcoRI restriction enzyme cutting site. PCR fragments were recovered by a kit (purchased from Shanghai Huashun Biological Engineering Co., Ltd.), and digested by BamHI and EcoRI (enzymes for each molecular biology used in the present disclosure were purchased from Dalian Takara Bio Inc.), and linked onto an expression vector pET-24a (Novagen, Art. No.: 69864-3) after being subjected to BamHI and EcoRI digestion, thus obtaining a recombinant plasmid pET-24a-W135.

The above positive clone was subjected to shaking culture in a 500 ml LB medium with a 100 ug/ml kanamycin sulfate (Sangon Biotech, hereinafter referred to as, Sangon, Art. No.: KB0286) at 37° C. till OD600=1.0 around, then the remaining product was induced by IPTG (Sangon, Art. No.: IB0168) having a final concentration of 0.5 mM for 4 h at 37° C. The culture was centrifuged for 20 min at 4° C., 5000 g to collect bacterial cells, and bacterial cells in per liter of bacteria solution were resuspended by 20 ml a lysis buffer (50 mMTirs-HCl, pH=8.0, 1 mM EDTA, 100 mM NaCl), and ultrasonicated; The culture was centrifuged for 20 min at 4° C., 12000 g, and identified by SDS-PAGE electrophoresis, the majority of the target proteins were distributed in supernatant of the lysis solution. The supernatant was collected, and dropwisely added with a saturated ammonium sulfate solution (Guangdong Guanghua Chemical Reagents Company, Art. No.: 7783-202, pH was adjusted to 7.4) till ammonium sulfate had a final concentration of 25%, standing for 30 mm at 4° C.; centrifuged for 20 min at 4° C., 12000 g to collect supernatant, and the supernatant was continuously dropwisely added with a saturated ammonium sulfate solution till ammonium sulfate had a final concentration of 40%, standing for 30 mm at 4° C.; centrifuged for 20 min at 4° C., 12000 g to collect precipitate, then the precipitate was dissolved by a 5 ml equilibration buffer (10 mM Na₂HPO₄, 1.8 mM KH₂PO₄, 140 mM NaCl, 2.7 mM KCl, 25 mM imidazole (Sigma-Aldrich, Art. No.: 15513), pH=8.0). An equilibration buffer with a 10-fold column bed volume was used for the equilibrium of a Ni-NTA affinity column (Qiagen, Art. No.: 30210), then a protein sample was added, and the unbound protein was washed by the equilibration buffer with a 10-fold medium volume; the target protein was eluted by a 5-fold-volume elution buffer (20 mM Na₂HPO₄, 300 mM NaCl, 250 mM imidazole, pH=8.0), and imidazole was dialyzed, and a protein concentration was measured, and the protein was stored at −20° C. for further use.

(2) Preparation of an HCV-AgII labeled antigen: by means of genetic engineering, a lot of molecular biology analysis software were used to analyze and screen out HCV NS3, NS4, and a dominant epitope segment of the core antigen, and the sequence was SEQ ID No. 2 (named W102); and the codons were optimized, and primers were designed (W102-F (SEQ ID No. 6):CGCGGATCCATGTCTACCAACCCGAAACCG; W102-R (SEQ ID No. 7):CCGGAATTCAGCGATCAGACGGTTCATCCAC) to amplify a DNA segment corresponding to W102, and the forward primer respectively carried a BamHI restriction enzyme cutting site, and the reverse primer carried an EcoRI restriction enzyme cutting site. PCR fragments were recovered by a kit (purchased from Shanghai Huashun Biological Engineering Co., Ltd.), and digested by BamHI and EcoRI (enzymes for each molecular biology used in the present disclosure were purchased from Dalian Takara Bio Inc.), and linked onto an expression vector pGEX-6P-I (Phamacia, Art. No.: 27-4597-01) after being subjected to BamHI and EcoRI digestion, thus obtaining a recombinant plasmid of the labeled antigen of the present disclosure, hereinafter referred to as, 6P-W102.

The above positive clone was inoculated in a 500 ml LB medium containing 100 ug/ml ampicillin sodium (Sangon Biotech, Art. No.: A0339) for shaking culture at 37° C. till OD600=1.0 around, then the remaining product was induced by IPTG having a final concentration of 0.5 mM for 4 h at 37° C. The culture was centrifuged for 20 min at 4° C., 5000 g to collect bacterial cells, and bacterial cells in per liter of bacteria solution were resuspended by 20 ml a blood lysis buffer (50 mMTirs-HCl, pH=8.0, 1 mM EDTA, 100 mM NaCl), and ultrasonicated; The culture was centrifuged for 20 min at 4° C., 2000 g, and identified by SDS-PAGE electrophoresis, 80% of the target proteins were distributed in supernatant of a lysis solution. The supernatant was collected, and dropwisely added with a saturated ammonium sulfate solution till ammonium sulfate had a final concentration of 15%, standing for 30 mm at 4° C.; centrifuged for 20 min at 4° C., 12000 g to collect supernatant, then the supernatant was continuously and dropwisely added with a saturated ammonium sulfate solution till ammonium sulfate had a final concentration of 45%, standing for 30 min at 4° C.; centrifuged for 20 min at 4° C., 12000 g to collect precipitate, and then the precipitate was dissolved by a 10 ml equilibration buffer (pH=7.3 PBS, 140 mM NaCl, 2.7 mM KCl, 10 mM Na₂HPO₄, 1.8 mM NaH₂PO₄). An equilibration buffer with a 10-fold column bed volume was used for the equilibrium of a GSTrap affinity column (Ainersham, Art. No.: 17-5130-02), then a protein sample was added, and the unbound protein was washed by the equilibration buffer with a 10-fold medium volume; the target protein was eluted by a 5-fold-volume elution buffer (50 mM Tris-HCl, and 10 mM reduced glutathione (Amreseo, Art. No.: 0399), pH=8.0), and a protein concentration was measured, and the protein was stored at −20° C. for further use.

Similarly, HCV-AgI-1 and HCV-AgII-1 were prepared, where HCV-Ag 1-1 had a 1201st-1490th amino acid sequence of NS3, and HCV-AgII-1 had a 1890th-1923rd amino acid sequence of NS4.

### Example 2 Preparation Process of HCV-Monoclonal Cells and Antibody Screening

2.1 Obtaining of C175 Gene Segments of an HCV Core Antigen Protein and Construction of PET32a-C175 Clone

By means of genetic engineering, a lot of molecular biology analysis software were used to analyze and screen out dominant epitope segments of the core antigen, and the sequence was SEQ ID No. 3 (named C175); and the codons were optimized, and primers were designed (C175-F (SEQ ID No. 8):CGCGGATCCATGTCTACCAACCCGAAACCG; C175-R (SEQ ID No. 9):CCGGAATTCAGAGAAAGAGCAACCCGGCA) to amplify a DNA segment corresponding to C175; a PCR product was taken and identified by 1.5% agarose gel, an specific band about 500 bp could be seen and corresponded to the size of expected 525 bp, and the target band was cut off for recovery. The fragment digested by double enzymes BamHI and EcoRI was linked into a PET32a vector digested by double enzymes BamHI and EcoRI, then transformed into a BL21(DE3) strain and identified by PCR, a recombinant plasmid PET32a-C175 was subjected to sequencing to prove whether a gene C175 was inserted into the vector correctly without any base or amino acid mutation, thus ensuring a correct reading frame.

2.2 Prokaryotic Expression and Purification of a Fusion Protein C175

Based on the above method, the genetically engineered bacteria containing the recombinant plasmid PET32a-C175 were induced by IPTG having a final concentration of 0.25 mM for 4 h. The result shows that there was an band of induced expression about 33 KD in the induced sample. The fusion protein was mainly expressed in a soluble form, and the molecular weight thereof accorded with the theoretical molecular mass. The remaining bacteria were ultrasonicated and centrifuged to collect supernatant, and the supernatant was subjected to Ni-affinity chromatography to obtain the fusion protein. A portion of the obtained fusion protein was reserved for further use, and another portion was digested by enterokinase to remove an N-terminal fusion protein, and reverse affinity chromatography was performed to obtain a non-fusion target protein, stored for further use. The protein sample was subjected to SDS-PAGE gel analysis; the fusion protein sample was named C175-A, about 33 KD, and the non-fusion protein sample was named C175-B, about 20 KD; and the purified target protein had a purity of 90% above.

2.3 Antigenicity of HCV Core Antigens C175-A and C175-B

Purified target proteins C175-A and C175-B were respectively coated onto an ELISA plate to detect the HCV positive quality control serum by an indirect ELISA method; the result showed that 8 copies of quality control serum had better reactivity to two proteins and showed positive reaction; C175-A had a mean value of 1.090, and C175-B had a mean value of 1.219; 8 copies of non-HCV positive clinical serum for parallel comparison showed negative reaction, C175-A had a mean value of 0.025, and C175-B had a mean value of 0.014; by making a comparison between C175-A and C175-B, C175-B had slightly better reactivity. Directed to the result, researchers may consider that the assisted expression of the fusion protein PET32a and low temperature induction rendered the HCV core antigen protein to have better antigenicity.

As shown in the table below: Reactivity of the recombinant HCV core antigens C175-A and C175-B

ELISA of the recombinant HCV antigens C175-A and C175-B

| Positive | P1 | P2 | P3 | P4 | P5 | P6 | P7 | P8 |
|---|---|---|---|---|---|---|---|---|
| C175-A | 0.232 | 0.541 | 0.876 | 1.023 | 1.325 | 1.275 | 1.579 | 1.872 |
| C175- B | 0.415 | 0.587 | 0.965 | 1.334 | 1.397 | 1.382 | 1.657 | 2.013 |
| Negative | N1 | N2 | N3 | N4 | N5 | N6 | N7 | N8 |
| C175-A | 0.032 | 0.024 | 0.018 | 0.021 | 0.023 | 0.028 | 0.017 | 0.034 |
| C175-B | 0.011 | 0.012 | 0.007 | 0.015 | 0.02 | 0.018 | 0.005 | 0.023 |

By comparison, C175-A and C175-B antigens had good reactivity to the HCV antibody positive serum; overall, the reactivity of C175-B was higher than C175-A; therefore, C175-B was selected to immunize mice.

3 Immunization of Mice by a Recombinant Antigen

1 ml C175-B antigen was taken and mixed with equivalent amount of freund's complete adjuvant, then the mixture was multi-injected into BALB/c mice subcutaneously/abdominally, and immunity was enhanced by injected abdominally 14 d after the primary immunization, and after injecting 4 injections for enhancing immunization, blood sampling was performed on the tail for titer determination, and the titer was up to the fusion requirements. Spleen was taken out under aseptic conditions for fusion use 3 d after mice were immunized for the last time.

3.1 Preparation of a Hybridoma Cell Line

(1) Preparation of Feeder Cells Peritoneal macrophage of BALB/c mice served as feeder cells. BALB/c mice were sacrificed by cervical dislocation 1 d before fusion, and completely soaked by 75% ethyl alcohol, then skin of abdomen was opened by scissors under sterile operation in a super clean bench to expose peritoneum, and 5 mL RPMI 1640 basic culture solution was injected abdominally with an injector, and the peritoneum was washed repeatedly, and the washing fluid was recycled, then the obtained peritoneum was centrifuged for 5 min at 1000 rpm to preserve precipitate, then resuspended by a RPMI 1640 screening culture solution (a RPMI 1640 complete culture solution containing HAT), then cell concentration was adjusted to 1×10⁵/mL, then cells were added to a 96-well plate with 150 μL/well, and cultured overnight at 37° C., and 5% C02.

(2) Preparation of Immune Spleen Cells Spleen was taken out under aseptic conditions for fusion use 3 d after mice were immunized for the last time, and put on a plate, and washed for once by a RPMI 1640 basic culture solution, then put on a nylon net of a small beaker for grinding and filtering, and made into a cell suspension. The cell suspension was centrifuged to discard supernatant, and resuspended by a RPMI 1640 basic culture solution, and the operation was repeated for three times for counting.

(3) Preparation of Myeloma Cells

Mice myeloma cells Sp2I0 (stored by Fapon Biotech Inc.) were screened by 8-azaguanine, then cultured to a logarithmic phase, and two big bottles were taken and made into a cell suspension, and the cell suspension was centrifuged to discard supernatant, and resuspended by a RPMI 1640 basic culture solution, and the operation was repeated for three times for counting.

(4) Cell Fusion and HAT Selection of Hybridoma

Myeloma cells were mixed with immune spleen cells according to a ratio of 1:10, and washed for once with a RPMI 1640 basic culture solution in a 50 mL plastic centrifugal tube, and centrifuged for 8 min at 1200 rpm. Supernatant was discarded, and cells were mixed evenly, and slowly added with 1 mL 50% PEG1500 for fusion, 1 min after fusion, 15 mL RPMI 1640 basic culture solution was added to terminate cell fusion. Cells were centrifuged for 5 min at 1000 rpm. Supernatant was discarded, and cells were slightly suspended with 50 mL RPMI 1640 screening culture solution, and divided equally onto 10 pieces of 96-well plates having feeder cells, 50 μL/well, and cultured at 37° C., 5% CO₂. When cells were cultured to the 6th day, HT culture solution (a RPMI 1640 complete culture solution containing HT) was changed for twice.

3.2 Screening of Antibodies of Anti-HCV Core Antigen Protein C175

Core antigens C175-A and C175-B were coated on an ELISA plate overnight at 4° C., then blocked by 0.02 M pH=7.2 PBS containing 10% fetal bovine serum or 1% skim milk powder, 0.15 ml/well for 2 h at 37° C.; cells were added to culture supernatant for 30 min at 37° C., then 30 min later, goat-anti-mouse IgG labeled by horseradish peroxidase (produced by Fapon Biotech Inc., Art. No.: BA-PAB-MU0001) 2000-fold diluted was added at 37° C., 30 min later, 100 μL pH=5.0 citric acid-phosphate buffer containing 0.1% (M/V) o-phenylenediamine and 0.1% (V/V) hydrogen peroxide was added per well at 37° C. for 15 min, then 50 μl dilute sulphuric acid solution was added per well, and absorbance at 450 nm was measured. A RPMI 1640 complete culture solution served as a negative control, when a ratio of a measured value to a control value was ≥2.0, it was positive cell well.

3.3 Construction of Monoclonal Antibody Cell Strains of Anti-HCV Core Antigen Protein

Cells were fused for 3 times and obtained 12 cell strains stably secreting anti-HCV core antigen 175B recombinant protein monoclonal antibodies in total, and the titer was within 10⁵-10⁷. The anti-HCV core antigen monoclonal antibodies were subjected to identification and classification of monoclonal antibodies by ELISA, where 6 monoclonal antibodies 3C-28, 11C-13, 14C-1, 1D-9, 8H-53, and 5G-28 were type IgG1; and 6 monoclonal antibodies 14C-77, 4G-19, 5B-36, 8D-73, 3G-42, and 2H-49 were type IgG2.

3.4 Epitope Identification of Anti-HCV Core Antigen McABs

Cells were fused for 3 times and obtained 21 cell strains stably secreting anti-HCV core antigen 175B recombinant protein monoclonal antibodies in total, and the titer was within 10⁵-10⁷.The anti-HCV core antigen monoclonal antibodies were subjected to identification and classification of monoclonal antibodies by ELISA, where 6 monoclonal antibodies 3C-28, 11C-13, 14C-1, 1D-9-10, 8H-53, and 5G-28 were type IgG1; and 6 monoclonal antibodies 14C-77, 3F-41, 58-36, 8D-73, 7C-14-9, 2H-49, and 12F-19 were type IgG2a; and monoclonal antibodies 4D-19, 3C-7, 2D-32, 5G-12, 6F-78, 6G-5-1, and 15D-8 were type IgG2b.

3.4 Epitope Identification of Anti-HCV Core Antigen McABs

8 HCV short peptide antigens A1-A8 were respectively coated on micro-wells, PBS+20% NBS served as a diluent to dilute monoclonal antibodies to a concentration of primary antibodies, 1 ug/mL; goat-anti-mouse IgG served as a second antibody, and epitopes of the monoclonal antibodies were determined according to the reaction condition of each monoclonal antibody to different antigens.

The result was shown in the table below: the monoclonal antibodies prepared by the C175-B antigen could identify 5 epitopes; there is no monoclonal antibody to identify C70-100 and C120-C175; where the number of monoclonal antibodies identifying C17-35, C55-72 and C95-117 epitopes was up to the maximum, there were 5 monoclonal antibodies to identify C17-35, 6 monoclonal antibodies to identify C60-72 (C55-72 via further verification by analysis), and 7 monoclonal antibodies to identify C100-120aa (C95-117 via further verification by analysis). It can be seen from the above epitope identification that the major epitopes identified by antibodies were distributed into three segments C17-35, C55-72, and C95-117, moreover, the three segments had best reactivity to the antigen.

### Epitope Identification of the HCV-Core Monoclonal Antibody

Epitope identification of the HCV-core monoclonal antibody

| | Monoclonal | A1 | A2 | A3 | A4 | A5 | A6 | A7 | A8 | Scope of the |
|---|---|---|---|---|---|---|---|---|---|---|
| No. | antibody No. | C1-25 | C17-35 | C30-49 | C41-59 | C55-72 | C70-100 | C95-117 | C120-175 | epitope identification |
| 1 | 3C-28 | − | + | + | − | − | − | − | − | 26-35aa |
| 2 | 1D-9-10 | − | − | − | − | + | − | − | − | 60-72aa |
| 3 | 7B-18 | − | − | − | − | − | − | + | − | 100-120aa |
| 4 | 4G-19 | − | + | − | − | − | − | − | − | 28-35aa |
| 5 | 12F-19 | − | + | − | − | − | − | − | − | 28-35aa |
| 6 | 8H-53 | − | − | − | − | − | − | + | − | 100-120aa |
| 7 | 14C-77 | − | − | − | − | + | − | − | − | 60-72aa |
| 8 | 8D-73 | − | − | − | − | + | − | − | − | 60-72aa |
| 9 | 3C-7 | − | − | − | + | − | − | − | − | 35-42aa |
| 10 | 6F-78 | − | − | | − | − | − | + | − | 100-120aa |
| 11 | 5B-36 | − | − | − | − | + | − | − | − | 60-72aa |
| 12 | 5G-12 | − | − | − | − | + | − | − | − | 60-72aa |
| 13 | 11C-13 | − | − | − | − | − | − | + | − | 100-120aa |
| 14 | 14C-1 | − | + | − | − | − | − | − | − | 28-35aa |
| 15 | 5G-28 | − | − | + | − | − | − | − | − | 35-42aa |
| 16 | 2H-49 | − | − | − | − | − | − | + | − | 100-120aa |
| 17 | 2D-32 | − | + | − | − | − | − | − | − | 26-35aa |
| 18 | 6G-5-1 | − | − | − | − | + | − | − | − | 60-72aa |
| 19 | 3F-41 | + | − | − | − | − | − | − | − | 1-25aa |
| 20 | 15D-8 | − | − | − | − | − | − | + | − | 100-120aa |
| 21 | 7C-14-9 | − | − | − | − | − | − | + | − | 100-120aa |

3.5 Antibody pairing Screening:

21 monoclonal antibodies were subjected to an orthogonal experiment by an ELISA sandwich method. To detect the dilution, screening high-sensitivity compatible monoclonal antibodies by the C175B core antigen; magnetic beads-coated by 30-28, 140-1, 6F-78, 11C-13, 15D-8, and 3G-42 were paired with AE-labeled 14C-77, ID-9-10, 2H-49, and 8D-73 to show that 20 groups of monoclonal antibodies had good compatibility, capable of detecting 97 pg/ml; where 48.5 ng/mL could be detected when 140-1, 6F-78 and 11C-13 were paired with AE-labeled 1D-9-10, 14C-77, 2H-49, and 8D-73, showing that the highest sensitivity of 48.5 ng/mL could be detected for 4 groups of monoclonal antibodies. Moreover, it can be also seen that 14-77 had high affinity when used for labeling terminals and other antibodies, and could be reacted with 20 monoclonal antibodies, and had the maximum pairing success rate.

See the table below

Table of Data analysis on the minimum limit of detection (LOD) of the
recombinant antigen by cross pairing with 24 groups of antibodies

| | Reactivity of the | | | | | | | | |
|---|---|---|---|---|---|---|---|---|---|
| | C175B | | | | | | | | |
| Cross pairing | recombinant | | | | | | | | |
| Magnetic bead | antigen | | | | | | | | |
| coated with | AE-labeled | | | | | | | | |
| antibody | antibody Epitope | 55-72aa | 55-72aa | 55-72aa | 55-72aa | 95-117aa | 95-117aa | 55-72aa | 55-72aa |
| 3C-28 | 17-35aa | * | / | * | / | * | / | * | / |
| 14C-1 | 17-35aa | * | * | * | * | * | * | * | * |
| 6F-76 | 95-117aa | * | * | * | * | * | * | * | * |
| 11C-13 | 95-117aa | * | * | * | * | * | * | * | * |
| 15D-8 | 95-117aa | * | / | * | / | * | / | * | / |
| 3G-42 | 95-117aa | * | * | * | * | * | * | * | * |

Remarks: / denotes no reaction after pairing, * denotes that the C175B antigen had better reactivity to two groups of antigen concentration (97 ng/ml and 48.5 ng/ml).

The pairing result indicated that epitopes C17-35 and C95-117 had activity superior to others when used for coating; epitopes C55-72 had activity superior to others when used for labeling; the combination effect of epitopes C95-117 and C55-72 was superior to the combination of C17-35 pairing to C55-72 in sensitivity.

3.6 Screening of Natural Positive Samples of the Core Antigen

78 samples (PCR positive and antibody positive) were collected in this laboratory; HCV core antigen detection kits purchased from Shandong Laibo Biotechnology Co., Ltd. were used to detect the positive core antigens in the 78 serum samples. The result showed that in the 78 serum samples, 47 samples had S/CO greater than the critical value, where the most of the serum samples had low reactivity, only 10 samples had S/CO greater than 5, and others were distributed within 1.0-5 of S/CO, indicating that the core antigen had a very low content in serum, additionally, a portion of may be probably neutralized by antibodies. Therefore, higher-activity antibody pairing was required to improve the detection sensitivity of the core antigen, and these core antigen positive samples were used in the subsequent process for screening the pairing monoclonal antibodies with high reactivity to natural core antigen positive samples.

3.7 Screening of Compatible Monoclonal Antibodies Having a High Detection Rate to Natural Core Antigen Positive Samples by a Magnetic Affinity Immunoassay Platform of a Double-Antibody Sandwich Method.

We could not predict the detection rate of the screened antibody pairs to the core antigen positive samples in; therefore, antibodies 3C-28, 14C-1, 11C-13, 15D-8, and 3G-42 were respectively coated on magnetic beads and then paired with AE-labeled 14C-77, ID-9-10, 2H-49, and 8D-73, there were 20 groups of compatible monoclonal antibodies; 10 RNA-positive groups were picked; 5 groups having S/CO greater than 5 and 5 groups having S/CO greater than 1-4 were selected to survey the reaction situation to the natural positive samples: the detection rate of core positive samples via the cross-paired monoclonal antibody combination.

Reactivity to 10 core antigen positive samples

| | pairing | | | | |
|---|---|---|---|---|---|
| Cross | Labeled | 1D-9-10- | 14C-TT- | 2H-49- | 8D-T3- |
| Envelope | antibody | AE | AE | AE | AE |
| antibody | Epitope | C55-72aa | C55-72aa | C95-117aa | C55-72aa |
| 3C-28 | 17-35aa | 6 copies | 8 copies | 5 copies | 6 copies |
| magnetic bead | | | | | |
| 14C-1 | 17-35aa | 6 copies | 8 copies | 4 parts | 8 parts |
| magnetic bead | | | | | |
| 6F-78 magnetic | 95-117aa | 7 copies | 9 copies | 6 copies | 6 parts |
| bead | | | | | |
| 11C-13 | 95-117aa | 8 copies | 10 copies | 5 parts | 8 copies |
| magnetic bead | | | | | |
| 15D-8 | 95-117aa | 7 copies | 8 copies | 5 parts | 7 copies |
| magnetic bead | | | | | |
| 3G-42 | 95-117aa | 8 copies | 8 copies | 4 parts | 8 parts |
| magnetic bead | | | | | |

Through the above screening, it can be seen that the monoclonal antibody pair of a combination of C95-117aa as an envelope paired with C55-72aa labeling has the maximum detection rate; 10 copies of serum were detected by 11C-13 paired with 14C-77-AE, superior to other pairing group, followed by a combination of C15-35aa paired with C55-72aa; the combination of C95-117aa as an envelope antibody paired with C95-117aa labeling and the combination of C17-35aa paired with 95-117aa had a minimum detection rate. The above result indicated that the dominant epitope pair for detecting the core antigen was mainly focused on the combination of C95-117aa as an envelope paired with C55-72aa labeling and the combination of C15-35aa paired with C55-72aa as an envelope antibody paired with C55-72aa labeling. Two groups of dominant epitope combinations were selected for the amplification of positive and clinical serum.

3.8 Comparison on the Amplification of the Positive and Clinical Negative Serum in the Two Groups of Dominant Epitope Combinations

Comparison on sensitivity and specificity

| | | | Copies of the | | Copies of | |
|---|---|---|---|---|---|---|
| | | | positive | | the false | |
| | | | samples | | positive | |
| | | | detected | | samples | |
| | | | in the | | detected in | |
| | | | 48 core | | the 300 | |
| | | | antigen | | clinical | |
| Envelope | | Labeled | positive | Detection | negative | Specificity |
| antibody | Epitope | antibody | samples | rate (%) | samples | (%) |
| 3C-28 | 17-35aa | 14C-77-AE | 40 copies | 83.30% | 3 | 99% |
| magnetic | | | | | | |
| bead | | | | | | |
| 14C-1 | 17-35aa | 14C-77-AE | 42 copies | 87.50% | 4 | 98.60% |
| magnetic | | | | | | |
| bead | | | | | | |
| 6F-78 | 95-117aa | 14C-77-AE | 46 copies | 95.80% | 2 | 99.30% |
| magnetic | | | | | | |
| bead | | | | | | |
| 11C-13 | 95-117aa | 14C-77-AE | 48 copies | 100% | 1 | 99.60% |
| magnetic | | | | | | |
| bead | | | | | | |
| 15D-8 | 95-117aa | 14C-77-AE | 44 copies | 91.6% | 1 | 99.50% |
| magnetic | | | | | | |
| bead | | | | | | |
| 3G-42 | 95-117aa | 14C-77-AE | 46 copies | 95.8% | 2 | 99.30% |
| magnetic | | | | | | |
| bead | | | | | | |

Conclusion: by comparison, the combination of 11C-13 envelope antibody paired with 14C-77 labeling having a consistent detection rate with the Shandong Laibo kit was screened, and the specificity also satisfied requirements. Other pairing had the problem of missing of partial low-value samples, which showed that the combination of C95-117aa envelope antibody paired with C55-72aa labeling was the optimum, and the combination of 11C-13 envelope antibody paired with 14C-77 labeling was superior to other pairing in sensitivity and specificity. Therefore, such a pairing was selected for the combined detection combination, thus achieving the preparation of a combined detection kit by a staggered epitope way.

Moreover, monoclonal antibodies specifically binding to a sequence in a 95-117 region and a sequence in a 55-72 region were purchased (HCV-Core-McAb23 and HCV-Core-McAb19were purchased from Fapon Biotech Inc.; the HCV-Core-McAb23 monoclonal antibody specifically bound to the sequence of the 95-117 region in the HCV core antigen; and the HCV-Core-McAb19 monoclonal antibody specifically bound to the sequence of the 55-72 region in the HCV core antigen). The antibodies with the highest sensitivity to C175-B and the reactivity of 10 natural samples positive to HCV-core antigens were measured. The result indicated that 10 natural samples were detected out via all the combinations of antibodies 2C-18, 3D-10, 5G-22, and HCV-Core-McAb23 directed against the epitope of the 95-117 region and 6G-15, 7H-3, and HCV-Core-McAb19 directed against the epitope of the 55-72 region; the reactivity to the core antigen was high, the detection rate of the natural HCV-core antigen positive samples was high, and the negative serum had good background. The above result also indicated that the detection sensitivity and specificity of the antibody combination directed against amino acids in 95-117aa and 55-72aa epitopes were superior to the pairing directed against other epitope antibodies. In the following experiment, an antibody 11C-13 as an envelope antibody/14C-77-AE pairing was selected for the experiment. 11C-13 was named HCV-AbI and 14C-77-AE was named HCV-AbII for the combined detection pairing.

### Example 3 Preparation of Magnetic Bead-Labeled Antigens and Antibodies

1) 10 mg carboxyl magnetic beads (Merk EM1-100/40 carboxyl magnetic beads) were taken and washed for 4 times with a activation buffer (100 mM MES, pH=5.5), 10 mL each time, and finally added with 8 mL activation buffer for ultrasonic dispersion. About NHS (10 mg) and EDC (5 mg) were weighed, (NHS (N-hydroxysuccinimide was purchased from Thermo, model: 24510) and EDC was purchased from Thermo, model: 22891), and respectively dissolved into 10 mg/mL and 1 mg/mL, then added with 1 mL NHS solution and 1 mL EDC solution, and mixed evenly, and subjected to rotary for 10 min at 30 rpm and room temperature.

2) Magnetic separation was performed and supernatant was discarded without washing, and 9 mL crosslinked buffer (the same as the activation buffer: 100 mM MES, pH=5.5) was added directly for ultrasonic dispersion; the activated magnetic beads were divided into two parts; one part of 4.5 ml activated magnetic beads were taken and added to HCV-AbI 0.5 mL (4.0 mg/mL), and another part of 4.5 ml magnetic beads were added to 0.5 ml HCV-AgI (passed a Zeba spin desalting column before adding, and purchased from Thermo, model: 89891) for rotary (30 rpm) reaction for 4 h at room temperature.

3) The magnetic beads were washed with 10 mL cleaning solution for twice; 10 mL blocking buffer (containing 0.5% BSA) was added for rotary (30 rpm) reaction for 4 h at room temperature.

4) The magnetic beads were washed with 10 mL cleaning solution for 3 times; finally, 5 mL magnetic bead preservation liquid (25 mM MES+150 mM NaCl+0.2% (w/v) Casein+1 mM EDTA+5% (v/v) NBS+0.2% Proclin-300) was added respectively for resuspending to a final concentration of 10 mg/mL (solid content), stored at +2° C. to +8° C.

### Example 4 Preparation of Avidin (SA) Coupling with Acridinium Ester (AE)

Example: The purified SA having a purity greater than 90% was taken and put to a dialysis bag for dialysis with 20 m MPB (pH=7.4) for 4 h; according to a ratio, AE (acridinium ester) was added for coupling (AE was purchased from Heliosense NSP-SA-NHS, model: 199293-83-9) and labeling for 10 min, and subsequently dialyzed for 4 h continuously, and the remaining solution was sucked out from the dialysis bag, and 50% glycerin was added for preservation at −20° C. for further use.

### Example 5 Preparation of n Biotinylated HCV Antigen In Vitro

1) sulfo-NHS-LC-biotin was used for labeling an amino of HCV antigen for description herein, and the labeling procedure was as follows:

2) 1 mg HCV-AgII antigen was taken and dialyzed by a buffer (100 mM PB+150 mM NaCl, pH=7.2) overnight;

3) biotin solution: 2.2 mg sulfo-NHS-LC-biotin were dissolved into 0.4 ml ultrapure water, and 143 μl biotin was taken and added to the above dialyzed antigen;

4) a protein solution was mixed with the biotin solution according to a molar ratio of 1:50, and crosslinked for 2 h at 0-4° C.;

5) the reaction solution was dialyzed in a buffer containing 0.05% SDS PB (100 mM PB, pH=7.2) to remove free biotin;

and 6) glycerin having a final concentration of 50% was added to the reaction solution for storage at −20° C. for further use.

### Example 6 Preparation of a Biotinylated HCV Antibody In Vitro

1) 4 mg HCV-AbII was taken and dialyzed by a buffer (100 mM PB+150 mM NaCl, pH=7.2) overnight;

2) biotin solution: 2.2 mg sulfo-NHS-LC-biotin were dissolved into 0.4 ml ultrapure water, and 53 μl biotin was taken and added to the above dialyzed antibody;

3) a protein solution was mixed with the biotin solution according to a molar ratio of 1:20, and crosslinked for 2 h at 2° C.-8° C., and dialyzed in a buffer containing 0.05% SDS PB (100 mM PB, pH=7.2) to remove free biotin;

and 4) glycerin having a final concentration of 50% was added to the reaction solution for storage at −20° C. for further use.

### Example 7 Lysis Solution for Detecting the Antigen-Antibody Combination Detection

1) 10-100 mM, preferably, 20 mM PB phosphate buffer was selected;

2) denaturant: SDS had a concentration of 0.5%-1%, preferably, 0.8%.

3) surfactant: NP-40 had a concentration of 0.5%-1%, preferably, 0.5%. TRITONX-100 and TWEEN-20 were added to 0.5%-1%, preferably, 0.5%.

4) BSA protective protein: the concentration was 0.5%-1%, preferably, 1%.

5) ammonium sulfate had a concentration of 1%-2.5%, preferably, 1%.

6) absolute ethyl alcohol: the concentration may be 0.1%-10%, preferably, 1%.

Note: the above concentration ratio was a mass-volume ratio, and 1% denoted 1 g/100 mL

### Example 8 Preparation of a Reagent in the Kit

1. A magnetic bead working solution (preparation of a mixed solution of magnetic bead-labeled HCV-AbI and HCV-AgI): 10 mg/ml prepared HCV-AgI magnetic bead was diluted by a preservation liquid to 0.2 mg/ml, and 10 mg/ml HCV-AbI magnetic bead was diluted by a preservation liquid to 0.2 mg/ml, and the previous two groups of solution were mixed by a volume ratio of 1:2 for further use.

2. A biotin working solution (preparation of a mixed solution of biotinylated HCV-AbII-BIO and HCV-AgII-BIO):

the labeled HCV-AgII-BIO was diluted by a biotin diluent (containing 20 mM PB+150 mM NaCl+0.1% Casein-2Na+0.1% P300+0.1% mercaptoethanol) to 0.2 mg/ml; and HCV-AgII-BIO was diluted by an HCV biotin diluent (containing 20 mM PB+150 mM NaCl+0.1% Casein-2Na+0.1% P300+0.1% mercaptoethanol) to 0.2 mg/mL, then the above two diluent were mixed by a ratio of 1:2 (the biotin diluent contained 1:1000 mercaptoethanol), where a reductant may be DTT, mercaptoethanol, and the like, preferably, mercaptoethanol.

3. Preparation of an avidinylated label SA-AE:

SA-AE was diluted by a diluent, 200 mM HEPES+0.5% BSA+0.1% sodium azide to 0.5 pg/ml for further use, and 0.5 μg/ml blocker was added.

4. Preparation of a lysis solution.

5. Preparation of a 20× cleaning solution, and the cleaning solution was diluted to 1× for further use.

6. Preparation of negative/positive quality control substances

7. Preparation of an triggers.

A kit 1 was prepared by the above steps.

Similarly, HCV-AgI and HCV-AgII were replaced with HCV-AgI-I and HCV-AgII-1 in the kit to prepare a kit 2.

### Example 9: Operation Process

1. Preparation of a detection reagent;

2. 50 μl magnetic bead working solution (a mixed solution of antigen and antibody magnetic beads)+100 μl sample+50 μl biotin working solution (a mixed solution of antigen and antibody biotin) were added per well for reaction for 15 min in a thermostat at 37° C.; then 50 μl lysis solution was added for reaction for 15 min in the thermostat at 37° C., and washed for 4 times;

3. 200 μl avidinylated SA-AE was added per well for reaction for 10 min in a thermostat at 37° C., and washed for 4 times;

4. 100 μl triggers A and 100 μl triggers B were added to measure a luminance value with an automatic chemiluminiscence instrument, and the luminance value was compared to the critical value, thus judging as positive or negative.

TABLE 5
Data comparison of antigen-antibody combined detection and single detection; result
judgment: >1 denoted positive; and <1 denoted negative.
Test results of the kit 1

| | | | Test | Test | Test by the | Test by the |
|---|---|---|---|---|---|---|
| | | | result of | result of | combined | combined |
| | | | the | the | magnetic | magnetic |
| | | | magnetic | magnetic | bead | bead |
| | | | bead | bead | detection | detection |
| | | | detection | detection | antibody- | antibody- |
| | Serum | Serum | core | antibody | core | core |
| Positive | background | background | antigen | kit | antigen kit 1 | antigen kit 2 |
| sample No. | antigen | antibody | kit (s/co) | (s/co) | (s/co) | (s/co) |
| P1 | − | + | 0.04 | 15.17 | 16.25 | 16.51 |
| P2 | − | + | 0.04 | 11.65 | 10.6 | 10.84 |
| P3 | − | + | 0.12 | 12.89 | 13.51 | 13.29 |
| P4 | − | + | 0.025 | 9.46 | 10.04 | 10.11 |
| P5 | − | + | 0.04 | 14.36 | 13.04 | 13.20 |
| P6 | − | + | 0.05 | 17.03 | 18.1 | 18.03 |
| P7 | − | + | 0.07 | 15.84 | 16.96 | 16.87 |
| P8 | + | + | 2.33 | 9.26 | 16.3 | 16.22 |
| P9 | + | + | 4.56 | 12.64 | 15.69 | 15.06 |
| P10 | + | + | 5.24 | 15.5 | 18.5 | 18.27 |
| P11 | + | + | 4.63 | 10.25 | 16.13 | 15.83 |
| P12 | + | + | 6.18 | 5.63 | 14.25 | 14.05 |
| P13 | + | + | 3.05 | 6.25 | 13.25 | 12.94 |
| P14 | + | + | 1.54 | 9.56 | 12.54 | 12.38 |
| P15 | + | + | 2.98 | 15.73 | 18.1 | 17.53 |
| P16 | + | − | 4.56 | 0.051 | 5.46 | 4.86 |
| P17 | + | − | 10.53 | 0.062 | 11.45 | 11.23 |
| P18 | + | − | 6.12 | 0.016 | 8.15 | 7.98 |
| P19 | + | − | 5.26 | 0.032 | 5.42 | 5.36 |
| P20 | + | − | 3.64 | 0.047 | 3.79 | 3.83 |
| 200 pg/ml | + | − | 5.13 | 0.042 | 5.45 | 5.55 |
| core antigen | | | | | | |
| quality | | | | | | |
| control | | | | | | |
| substance | | | | | | |
| 100 pg/ml | + | − | 2.09 | 0.032 | 3.514 | 3.64 |
| core antigen | | | | | | |
| quality | | | | | | |
| control | | | | | | |
| substance | | | | | | |
| N1 | − | − | 0.042 | 0.021 | 0.044 | 0.041 |
| N2 | − | − | 0.021 | 0.015 | 0.020 | 0.017 |
| N3 | − | − | 0.015 | 0.040 | 0.015 | 0.013 |
| N4 | − | − | 0.035 | 0.012 | 0.028 | 0.031 |
| N5 | − | − | 0.016 | 0.018 | 0.013 | 0.015 |
| N6 | − | − | 0.015 | 0.028 | 0.016 | 0.014 |
| N7 | − | − | 0.029 | 0.027 | 0.026 | 0.022 |
| N8 | − | − | 0.017 | 0.039 | 0.024 | 0.019 |
| N9 | − | − | 0.077 | 0.032 | 0.081 | 0.052 |
| N10 | − | − | 0.023 | 0.033 | 0.022 | 0.029 |
| N11 | − | − | 0.029 | 0.041 | 0.027 | 0.021 |
| N12 | − | − | 0.065 | 0.037 | 0.057 | 0.043 |
| N13 | − | − | 0.045 | 0.032 | 0.044 | 0.040 |
| N14 | − | − | 0.017 | 0.025 | 0.018 | 0.024 |
| N15 | − | − | 0.031 | 0.044 | 0.029 | 0.033 |
| N16 | − | − | 0.066 | 0.029 | 0.072 | 0.060 |
| N17 | − | − | 0.033 | 0.033 | 0.028 | 0.031 |
| N18 | − | − | 0.025 | 0.045 | 0.028 | 0.037 |
| N19 | − | − | 0.059 | 0.046 | 0.058 | 0.046 |
| N20 | − | − | 0.026 | 0.035 | 0.022 | 0.028 |

Comparison data of the lysis solution:

| | | Sohlin control lysis | | |
|---|---|---|---|---|
| | | solution for | Novel lysis | Lysis solution for |
| | | combination | solution of the | single detection of |
| Sample | Background | detection | present disclosure | the core antigen |
| Original fold of | Ab+ | 7.32 | 16.56 | 2.68 |
| HCVP1 | | | | |
| HCVP1/20 | Ab+ | 3.99 | 10.98 | 1.32 |
| HCVP1/40 | Ab+ | 2.27 | 7.31 | 0.92 |
| HCVP1/80 | Ab+ | 0.96 | 11.25 | 0.46 |
| HCVP1/160 | Ab+ | 0.47 | 8. 56 | 0.15 |
| HCVP1/2000 | Ab+ | 0.19 | 3.32 | 0.10 |
| Original fold of | Ab+ | 8. 61 | 20.68 | 3.19 |
| HCVP2 | | | | |
| HCVP2/100 | Ab+ | 5.77 | 15.10 | 1.52 |
| HCVP2/200 | Ab+ | 2.93 | 16.72 | 0.91 |
| HCVP2/400 | Ab+ | 2.13 | 6.68 | 0.33 |
| HCVP2/1000 | Ab+ | 0.74 | 3.54 | 0.28 |
| HCVP2/4000 | Ab+ | 0.07 | 0.12 | 0.11 |
| Sera 462 | Ag+/Ab− | 1.82 | 2.11 | 2.28 |
| Sera 464 | Ag+/Ab− | 2.09 | 2.73 | 2.59 |
| LC1 | Ag−/Ab− | 0.66 | 0.14 | 0.15 |
| LC2 | Ag−/Ab− | 0.36 | 0.14 | 0.15 |
| LC3 | Ag−/Ab− | 0.36 | 0.15 | 0.16 |
| LC4 | Ag−/Ab− | 0.42 | 0.14 | 0.14 |

Data analysis table for the comparison on the effect of the lysis solution: (S/CO) result judgment: >1 denoted positive; and <1 denoted negative.

Conclusion: it can be seen from the above results that the adjusted lysis solution for the antigen-antibody combination detection had no influence on the detection of the antibody, and had lysis influences on the detection of the antigen, thus improving the detection rate of the antigen.

Therefore, the anti-HCV antigen monoclonal antibody of the present disclosure has no overlapped epitope, thus avoiding the problem that epitopes are difficultly staggered for the antigen-antibody combination detection. The anti-HCV antigen monoclonal antibody and HCV recombinant antigen of the present disclosure are free of cross reaction, thus free from influencing the activity and advantageously avoiding the problem of cross reaction and loss of the detection activity. The method and kit of the present disclosure remarkably shortens the window phase, reduces the risk of missing detection and workload, and lowers the manpower, instrument and reagent costs of the two methodologies when used for detection alone, thus improving the virus detection rate and sensitivity.

What is claimed is:

1. A hepatitis C virus detection kit, comprising a primary antibody and a second antibody for detecting a hepatitis C virus core antigen in a sample from a subject, wherein the primary antibody is directed against an epitope in a 95th-117th amino acid sequence of the hepatitis C virus core antigen or specifically binds to the 95th-117th amino acid sequence of the hepatitis C virus core antigen, and the second antibody is directed against an epitope in a 55th-72nd amino acid sequence of the hepatitis C virus core antigen or specifically binds to the 55th-72nd amino acid sequence of the hepatitis C virus core antigen.

2. The kit of claim 1, wherein the primary antibody is a capture antibody, the second antibody is a labeled antibody, or the primary antibody is a labeled antibody, and the second antibody is a capture antibody; preferably, the primary antibody is a capture antibody, and the second antibody is a labeled antibody.

3. The kit of claim 2, wherein the capture antibody is bound to a solid phase, and the labeled antibody is labeled by a detectable label.

4. The kit of claim 1, further comprising a primary antigen and/or a second antigen for detecting a hepatitis C virus antibody in a sample from a subject.

5. The kit of claim 4, wherein the primary antigen is a capture antigen, the second antigen is a labeled antigen; or the primary antigen is a labeled antigen, and the second antigen is a capture antigen.

6. The kit of claim 5, wherein the capture antigen is bound to a solid phase, and the labeled antigen is labeled by a detectable label.

7. The kit of claim 3, wherein the solid phase comprises magnetic particles, latex particles and a microtitration plate.

8. The kit of claim 1, comprising a virus lysis solution, for example, a phosphate buffer.

9. The kit of claim 8, wherein the virus lysis solution comprises 10-100 mM phosphate buffer, 0.5%-1% (m/v) denaturant, e.g., SDS, 0.5%-1% (m/v) surfactant, e.g., NP-40, TRITONX-100 and/or TWEEN-20, 0.5%-1% (m/v) protective protein, e.g., BSA, 1%-2.5% (mlv) ammonium sulfate, and 0.1%-10% (mlv) absolute ethyl alcohol.

10. The kit of claim 1, wherein the sample comprises a healthy or pathological biological tissue, cell or body fluid, for example, a blood sample, for example, plasma, serum, blood products, for example, seminal fluid or vaginal secretion, and wherein the hepatitis C virus comprises HCV genotypes I/1a, II/1b, III/2a, IV/2b, V/3a and VI/3b.

11. A method for detecting a hepatitis C virus, the method comprising: contacting a sample from a subject with the primary antibody and the second antibody, wherein the primary antibody is directed against an epitope in a 95th-117th amino acid sequence of the hepatitis C virus core antigen or specifically binds to the 95th-117th amino acid sequence of the hepatitis C virus core antigen; and the second antibody is directed against an epitope in a 55th-72nd amino acid sequence of the hepatitis C virus core antigen or specifically binds to the 55th-72nd amino acid sequence of the hepatitis C virus core antigen.

12. The method of claim 11, wherein the method further comprises: contacting the sample from the subject with the primary antigen and/or the second antigen from hepatitis C virus; the primary antigen and/or the second antigen may be, for example, a hepatitis C virus core antigen, E1, E2, NS2, NS3 NS4 and NS5, for example, the primary antigen and/or the second antigen originate from different positions of a same hepatitis C virus antigen, for example, a 7th-48th amino acid sequence from the hepatitis C virus core antigen, for example, a 7th-21st amino acid sequence and/or 29th-48th amino acid sequence from the hepatitis C virus core antigen; for example, the primary antigen and/or the second antigen comprises any one of the following amino acid fragments or a combination thereof: 1st-56th amino acids of an HCV core antigen, 1201st-1490th amino acids of NS3, a 1883rd-1925th amino acid sequence of NS4; 1st-35th amino acids of an HCV core antigen, 1223rd-1426th amino acids of NS3, a 1890th-1923rd amino acid sequence of NS4; for example, an amino acid sequence as shown in SEQ ID NO:1 and/or SEQ ID NO:2.

13. A method for preparing a reagent or kit for detecting a hepatitis C virus, wherein a primary hepatitis C virus core antigen and a second hepatitis C virus core antigen are used in preparation of antibodies, the primary hepatitis C virus core antigen comprises or consists of 55th-72nd amino acids of the hepatitis C virus core antigen; and the second hepatitis C virus core antigen comprises or consists of 95th-117th amino acids of the hepatitis C virus core antigen.

14. The method of claim 13, wherein the antibodies are monoclonal antibodies.

15. The method of claim 13, wherein the method comprises preparation of a kit for detecting a hepatitis C virus, wherein the kit comprises the antibody, and further comprises one or two antigens of hepatitis C virus; the one or two antigens may be, for example, a hepatitis C virus core antigen, E1, E2, NS2, NS3, NS4 and NS5, for example, different positions from a same hepatitis C virus antigen, for example, a 7th-48th amino acid sequence from the hepatitis C virus core antigen, for example, a 7th-21st amino acid sequence and/or 29th-48th amino acid sequence from the hepatitis C virus core antigen; for example, the one or two antigens comprise any one of the following amino acid sequence or a chimeric fragment thereof: 1st-56th amino acids of an HCV core antigen, 1201st-1490th amino acids of NS3, a 1883rd-1925th amino acid sequence of NS4; 1st-35th amino acids of an HCV core antigen, 1223rd-1426th amino acids of NS3, a 1890th-1923rd amino acid sequence of NS4; for example, an amino acid sequence as shown in SEQ ID NO:1 and/or SEQ ID NO:2.

16. The kit of claim 3, wherein the detectable label comprises fluorescence labeling, chromophore labeling, electron-dense labeling, chemiluminescent labeling, radiolabeling, enzyme labeling; for example, radioisotope, fluorophore, rhodamine and derivatives thereof, luciferase, fluorescein, horse radish peroxidase, alkaline phosphatase, β-galactosidase, glucoamylase, lysozyme, saccharides oxidase, glucose oxidase, galactose oxidase, glucose-6-phosphate dehydrogenase, biotin/avidin, spin labeling, phage labeling; for example, acridinium ester labeling; for example, wherein fluorescence labeling, e.g., acridinium ester labeling, is added by an adapter, e.g., biotin-avidin.

17. The kit of claim 4, wherein the primary antigen and/or second antigen is a hepatitis C virus core antigen, E1, E2, NS2, NS3, NS4 and NS5; for example, the primary antigen and/or second antigen originate from different positions of a same hepatitis C virus antigen.

18. The kit of claim 17, wherein, 1st-56th amino acids of the hepatitis C virus core antigen, for example, 1st-35th amino acids of the hepatitis C virus core antigen, for example, a 7th-48th amino acid sequence from the hepatitis C virus core antigen, for example, a 7th-21st amino acid sequence and 29th-48th amino acid sequence from the hepatitis C virus core antigen; for example, 1201st-1490th amino acids of NS3, a 1883rd-1925th amino acid sequence of NS4; 1223rd-1426th amino acids of NS3, and a 1890th-1923rd amino acid sequence of NS4; for example, the primary antigen comprises any one of the following amino acid fragments or a combination thereof: 1st-56th amino acids of an HCV core antigen, 1201st-1490th amino acids of NS3, a 1883rd-1925th amino acid sequence of NS4; and the second antigen comprises any one of the following amino acid sequence or a combination thereof: 1st-35th amino acids of an HCV core antigen, 1223rd-1426th amino acids of NS3, a 1890th-1923rd amino acid sequence of NS4; for example, an amino acid sequence as shown in SEQ ID NO:1 and/or SEQ ID NO:2.

19. The kit of claim 3, wherein the detectable label comprises fluorescence labeling, chromophore labeling, electron-dense labeling, chemiluminescent labeling, radiolabeling, enzyme labeling; for example, radioisotope, fluorophore, rhodamine and derivatives thereof, luciferase, fluorescein, horse radish peroxidase, alkaline phosphatase, β-galactosidase, glucoamylase, lysozyme, saccharides oxidase, glucose oxidase, galactose oxidase, glucose-6-phosphate dehydrogenase, biotin/avidin, spin labeling, phage labeling; for example, acridinium ester labeling; for example, fluorescence labeling, e.g., acridinium ester labeling, is added by an adapter, e.g., biotin-avidin.

20. The kit of claim 8, wherein, the virus lysis solution comprising a denaturant, a surfactant, a protective protein, ammonium sulfate and absolute ethyl alcohol.
